(19) United States
(12) Patent Application Publication
(10) Pub. No.: US 2014/0261515 A1
(43) Pub. Date: Sep. 18, 2014

(54) HAIR-CARE AGENTS HAVING POLYQUATERNIUM-71 AND CATIONIC SILICONES

(71) Applicant: Henkel AG & Co. KGaA, Duesseldorf (DE)

(72) Inventors: Manuela Mette, Kleinfeld (DE); Nicole Zuedel Fernandes, Hamburg (DE); Joerg Kahre, Leichlingen (DE)

(73) Assignee: Henkel AG & Co. KGaA, Duesseldorf (DE)

(21) Appl. No.: 14/287,294

(22) Filed: May 27, 2014

Related U.S. Application Data
(63) Continuation of application No. PCT/EP2012/072460, filed on Nov. 13, 2012.

(30) Foreign Application Priority Data
Nov. 29, 2011 (DE) 10 2011 087 270.1

(51) Int. Cl. A61K 8/891; A61K 8/34; A61Q 5/00; A61K 8/88; A61Q 5/12; A61Q 5/02

(52) U.S. Cl. CPC A61K 8/891; A61Q 5/12; A61Q 5/02; A61Q 5/002; A61K 8/88; A61K 8/342
USPC 132/202; 424/70.12; 510/122

(57) ABSTRACT

A hair-treatment agent includes an active-ingredient complex including polyquaternium-71 and at least one cationic silicone having at least three terminal amino-functional groups as essential constituents.

### FIELD OF THE INVENTION

The present invention generally relates to hair treatment agents including Polyquaternium-71 and cationic silicones.

### BACKGROUND OF THE INVENTION

The need exists to improve hair-care products further and to provide them with further advantageous properties. In particular, a care complex should be provided which ideally can also be used in combination with oxidizing agents and surfactant agents.

Environmental influences and oxidative hair treatments often lead to impaired combability of dry and wet hair. Furthermore, the gloss and moisture balance are disadvantageously affected by the attack on the external structure of the keratinic fibers. A further consequence of repeated treatments of keratinic fibers with surfactant and/or oxidative agents is marked regreasing of the keratinic fibers and a marked tendency towards increased formation of dandruff.

It is therefore desirable to reduce the side effects of environmental influences and of oxidative and surfactant hair treatments, preferably during the oxidative or surfactant hair treatment itself but also after the oxidative or surfactant hair treatment, without impairing the effectiveness of the oxidative or surfactant cosmetic, in particular with regard to color intensity, color-fastness, lightening performance or waving performance, and to prevent regreasing of the keratinic fibers and the increased formation of dandruff. In addition, the oxidative treatment of keratinous fibers, in particular human hair, should also be combined with the application of an effective protection of fibers from environmental influences, e.g. UV protection, in one application step in the form of a 2-in-1 product.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A hair treatment agent including in a suitable cosmetic carrier, based in each case on the overall composition of the agent, at least 0.01 to 10.0 wt. % Polyquaternium-71, and at least one cationic amino silicone having at least three terminal amino functional groups in a total quantity of 0.01 to 5.0 wt. %.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Surprisingly, it has now been found that the object of the invention is achieved to an excellent extent by a hair treatment agent, which includes an active substance complex including as essential ingredients at least Polyquaternium-71 and at least one cationic silicone with at least three terminal amino functional groups.

Hair treatment agents including this active substance complex lead to an improvement in brightness, gloss and moisture balance and to protection from oxidative damage and prevention of regreasing of keratinic fibers as well as to an increase in the wash resistance of dyed keratinic fibers, in particular human hair, and to a time delay in the formation of dandruff.

Hair treatment agents within the meaning of the present invention are, for example, hair shampoos, hair conditioners, conditioning shampoos, hair rinses, deep conditioners, hair masques, hair tonics, hair coloring shampoos or combinations thereof. In particular, the hair treatment agents according to the invention are understood to be hair-conditioning compositions, such as hair rinses, deep conditioners, hair masques, hair oils and lotions that are either leave-on products, i.e. remaining on the hair until the next hair wash, or rinse-off products, i.e. to be rinsed off again a few seconds to a few hours after application.

Combability is understood according to the invention to be both the combability of the wet fibers and the combability of the dry fibers.

Handle is defined as the tactility of a fiber collective, with the person skilled in the art feeling and evaluating the parameters of fullness and suppleness of the collective by sensory means.

Shaping is understood to be the ability to impart a change of shape to a collective of previously treated keratin-including fibers, in particular human hair. In hair cosmetics, the term “styling” is also used.

Restructuring within the meaning of the invention is understood to be a reduction of damage to keratinic fibers from a wide variety of influences. For example, restoring the natural strength plays an essential part here. Restructured fibers are distinguished by improved gloss, improved handle and greater ease of combing. In addition, they exhibit improved strength and elasticity. Moreover, successful restructuring can be detected physically as an increase in melting point compared with the damaged fiber. The higher the melting point of the hair, the stronger the structure of the fiber.

Washfastness within the meaning of the invention is the retention of the original dyeing with respect to shade and/or intensity when the keratinic fiber is exposed to the repeated influence of aqueous agents, in particular surfactant-including agents such as shampoos.

Furthermore, the compositions according to the invention including the active substance complex according to the invention are distinguished by a significantly improved state of the keratinic fibers in relation to the moisture balance of the keratinic fibers. Furthermore, the active substance complex according to the invention leads to a distinct protection of the keratinic fibers from the effects of heat, e.g. during blow-drying of keratinic fibers. The protection of the surface of keratinic fibers from the effect of heat is of great importance, particularly during the use of straighteners or hair dryers. Finally, it has surprisingly been found that the compositions according to the invention lead to a significantly delayed resoiling of the keratinic fibers. Moreover, the formation of dandruff on the scalp is significantly delayed.

An aqueous cosmetic carrier includes at least 50 wt. % water.

Aqueous-alcoholic cosmetic carriers within the meaning of the present invention are understood to be aqueous solutions including 3 to 70 wt. % of a C₁-C₆ alcohol, in particular methanol, ethanol or propanol, isopropanol, butanol, isobutanol, tert.-butanol, n-pentanol, isopentanols, n-hexanol, isohexanols, glycol, glycerol, 1,2-pentanediol, 1,5-pentanediol, 1,2-hexanediol or 1,6-hexanediol. The agents according to the invention can additionally include further organic solvents, such as e.g. methoxybutanol, benzyl alcohol, ethyl diglycol or 1,2-propylene glycol. All water-soluble organic solvents are preferred here. Water is particularly preferred.

The present invention therefore firstly provides a hair treatment agent including in a suitable cosmetic carrier—based in each case on the overall composition of the agent—
a) at least 0.01 to 10.0 wt. % Polyquaternium-71 and
b) at least one cationic amino silicone having at least three terminal amino functional groups in a total quantity of 0.01 to 5.0 wt. %.

When this combination is used, surprisingly good properties of the treated hair, in particular improved combability, improved gloss and improved elasticity, are obtained together with significantly increased wash resistance of dyed hair as well as longer durability at the same time as better reshaping performance in waving procedures, such as the water wave and permanent wave.

The first essential component is Polyquaternium-71.

This polymer can be obtained e.g. with the name Cola® Moist 300 P from Colonial Chemical Inc.

Polyquaternium-71 is used in a total quantity of 0.01 to 10.0 wt. %, preferably of 0.05 to 5.0 wt. %, more preferably of 0.1 to 3.0 wt. % and most preferably in quantities of 0.2 to 2.0 wt. %, based in each case on the total weight of the composition.

The second essential component of the active substance complex is a cationic aminosilicone. The cationic aminosilicones with at least three terminal amino functional groups have only been commercially available for a short time. These cationic silicone polymers are distinguished by the fact that they have a silicone backbone and optionally a polyether section and also at least one section with an ammonium structure. Examples of the preferred cationic silicone polymers within the meaning of the present invention are in particular the compounds with the INCI names: Silicone Quaternium-1, Silicone Quaternium-2, Silicone Quaternium-3, Silicone Quaternium-4, Silicone Quaternium-5, Silicone Quaternium-6, Silicone Quaternium-7, Silicone Quaternium-8, Silicone Quaternium-9, Silicone Quaternium-10, Silicone Quaternium-11, Silicone Quaternium-12, Silicone Quaternium-15, Silicone Quaternium-16, Silicone Quaternium-17, Silicone Quaternium-18, Silicone Quaternium-20, Silicone Quaternium-21, Silicone Quaternium-22 and Silicone Quaternium-2 Panthenol Succinate and Silicone Quaternium-16/Glycidyl Dimethicone Crosspolymer. Most preferred is in particular Silicone Quaternium-22. This raw material is marketed e.g. by Evonik with the trade name Abil® T-Quat 60.

The cationic amino functional silicone polymers are included in the compositions according to the invention in quantities of 0.01 to 5 wt. %, preferably in quantities of 0.05 to 5 wt. % and most particularly preferably in quantities of 0.1 to 5 wt. %. The best results of all are obtained with quantities of 0.1 to 2.5 wt. %, based in each case on the overall composition of the respective agent.

Furthermore, it is most preferred according to the invention if at least one amphoteric and/or zwitterionic surfactant is included in the compositions according to the invention. These ingredients possibly make a considerable contribution to stabilizing the viscosity and storage behavior in the compositions according to the invention.

Particularly suitable zwitterionic surfactants are the so-called betaines, such as the N-alkyl-N,N-dimethylammonium glycinates, e.g. cocoalkyl-dimethylammonium glycinate, N-acylaminopropyl-N,N-dimethylammonium glycinates, e.g. cocoacylaminopropyldimethylammonium glycinate, and 2-alkyl-3-carboxymethyl-3-hydroxyethylimidazolines, each with 8 to 18 C atoms in the alkyl or acyl group and cocoacylaminoethylhydroxyethylcarboxymethyl glycinate. A preferred zwitterionic surfactant is the fatty acid amide derivative known by the INCI name Cocamidopropyl Betaine.

Ampholytic surfactants (Tampho) are understood to be those surface-active compounds that are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids, each with about 8 to 24 C atoms in the alkyl group. Typical examples of amphoteric or zwitterionic surfactants are alkyl betaines, alkyl amidobetaines, aminopropionates, aminoglycinates, imidazolinium betaines and sulfobetaines.

Particularly preferred ampholytic surfactants are N-cocoalkyl aminopropionate, cocoacylaminoethyl aminopropionate and C₁₂-C₁₈ acylsarcosine. A particularly preferred compound is Coco Betaine.

These ingredients are used in quantities of 0.01 to 5.0 wt. %, based on the overall composition of the agent. Quantities of 0.05 to 5.0 wt. % are preferred. Particularly preferred are quantities of 0.1 to 5.0 wt. %, most preferably 0.3 to 3.0 wt. %.

The present invention therefore secondly provides a hair treatment agent including in a suitable cosmetic carrier—based in each case on the overall weight of the agent—
a) Polyquaternium-71 in a total quantity of 0.01 to 10.0 wt. %, and
b) at least one cationic aminosilicone having at least three terminal amino functional groups in a total quantity of 0.01 to 5.0 wt. % and
c) at least one surfactant selected from the zwitterionic and/or amphoteric surfactants in a total quantity of 0.01 to 5.0 wt. %.

To this most preferred backbone of ingredients it is furthermore possible to add all ingredients that are conventional in cosmetic compositions.

A further increase in the effects according to the invention is achieved by further cationic and/or amphoteric ingredients. These are in principle monomeric cationic or amphoteric ammonium compounds, monomeric amines, aminoamides, polymeric cationic ammonium compounds and polymeric amphoteric ammonium compounds. From the large number of possible cationic surfactants and/or cationic polymers, the following compounds have proved particularly suitable.

Cationic surfactants of the formula (Tkat1-1) are the first group of preferred cationic compounds.

[chemical structure]

In the formula (Tkat1), R1, R2, R3 and R4, each independently of one another, denote hydrogen, a methyl group, a phenyl group, a benzyl group, a saturated, branched or unbranched alkyl residue with a chain length of 8 to 30 carbon atoms, which can optionally be substituted with one or more hydroxy groups. A denotes a physiologically acceptable anion, e.g. halides such as chloride or bromide as well as methosulfates.

Examples of compounds of the formula (Tkat1) are lauryltrimethylammonium chloride, cetyltrimethylammonium chloride, cetyltrimethylammonium bromide, cetyltrimethylammonium methosulfate, dicetyldimethylammonium chloride, tricetylmethylammonium chloride, stearyltrimethylammonium chloride, distearyldimethylammonium chloride, lauryldimethylbenzylammonium chloride, behenyltrimethylammonium chloride, behenyltrimethylammonium bromide, behenyltrimethylammonium methosulfate.

Ester quats according to the formula (Tkat1-2) are further examples of cationic surfactants.

[chemical structure]

Here, the residues R1, R2 and R3 are each independent of one another and can be the same or different. The residues R1, R2 and R3 signify:
a branched or unbranched alkyl residue with 1 to 4 carbon atoms, which can include at least one hydroxyl group, or
a saturated or unsaturated, branched or unbranched or a cyclic saturated or unsaturated alkyl residue with 6 to 30 carbon atoms, which can include at least one hydroxyl group, or
an aryl or alkaryl residue, e.g. phenyl or benzyl,
the residue (—X—R4), with the proviso that no more than 2 of the residues R1, R2 or R3 can denote this residue.

The residue —(X—R4) is included at least 1 to 3 times.

X here denotes:
1) —(CH₂)ₙ— with n=1 to 20, preferably n=1 to 10 and particularly preferably n=1-5, or
2) —(CH₂—CHR5-O)ₙ— with n=1 to 200, preferably 1 to 100, particularly preferably 1 to 50, and particularly preferably 1 to 20 with R5 in the meaning of hydrogen, methyl or ethyl,
3) a hydroxyalkyl group with one to four carbon atoms, which can be branched or unbranched, and which includes at least one and no more than 3 hydroxy groups. Examples of —X— are: —CHOH, —CHCH₂OH, —CH₂CHOH, —COHCHOH, —CHOHCOH, —CHCHOHCH₃, —CH₂COHCH₃, —CH₂CHOHCH₂—, —C(CH₂OH)₂, —CH₂CHOHCH₂OH, —CH₂CH₂CHOH, —CH₂COHCH₃ and hydroxybutyl residues, wherein the bond of —X— to R4 starts from the free valency of the respective carbon atom
   and R4 denotes:
1) R6-O—CO—, where R6 is a saturated or unsaturated, branched or unbranched or a cyclic saturated or unsaturated alkyl residue with 6 to 30 carbon atoms, which can include at least one hydroxy group and which can optionally also be ethoxylated with 1 to 100 ethylene oxide units and/or 1 to 100 propylene oxide units, or
2) R7-CO—, where R7 is a saturated or unsaturated, branched or unbranched or a cyclic saturated or unsaturated alkyl residue with 6 to 30 carbon atoms, which can include at least one hydroxy group, and which can optionally also be ethoxylated with 1 to 100 ethylene oxide units and/or 1 to 100 propylene oxide units,
   and A denotes a physiologically acceptable organic or inorganic anion and is defined at this point in representation of all the structures described, including those below. The anion in all of the cationic compounds described is selected from the halide ions, fluoride, chloride, bromide, iodide, sulfates of the general formula RSO3⁻, where R has the meaning of saturated or unsaturated alkyl residues with 1 to 4 carbon atoms, or anionic residues of organic acids such as maleate, fumarate, oxalate, tartrate, citrate, lactate or acetate.

These products are marketed e.g. with the trademarks Rewoquat®, Stepantex®, Dehyquart®, Armocare® and Akypoquat®. The products Armocare® VGH-70, Dehyquart® F-75, Dehyquart® C-4046, Dehyquart® L80, Dehyquart® F-30, Dehyquart® AU-35, Rewoquat® WE18, Rewoquat® WE38 DPG, Stepantex® VS 90 and Akypoquat® 131 are examples of these ester quats.

Other particularly preferred compounds according to the invention of the formula (Tkat1-2) are included in formula (Tkat1-2.1), the cationic betaine esters.

[chemical structure]

R8 corresponds in its meaning to R7.

Particularly preferred are the ester quats with the trade names Armocare® VGH-70, as well as Dehyquart® F-75, Dehyquart® L80, Stepantex® VS 90 and Akypoquat® 131.

Quaternary imidazoline compounds are another group. The formula (Tkat2) illustrated below shows the structure of these compounds.

[chemical structure]

The residues R independently of one another each denote a saturated or unsaturated, linear or branched hydrocarbon residue with a chain length of 8 to 30 carbon atoms. The preferred compounds of the formula (Tkat2) include the same hydrocarbon residue for R in each case. The chain length of the residues R is preferably 12 to 21 carbon atoms. A denotes an anion as described above. Particular examples according to the invention are available e.g. with the INCI names Quaternium-27, Quaternium-72, Quaternium-83 and Quaternium-91. Most preferred according to the invention is Quaternium-91.

In a particularly preferred embodiment of the invention, the agents according to the invention also include at least one amine and/or cationized amine, in particular an amidoamine and/or a cationized amidoamine with the following structural formulae:

R1-NH—(CH₂)ₙ—N⁺R²R³R⁴A (Tkat3)

where R¹ signifies an acyl or alkyl residue with 6 to 30 C atoms, which can be branched or unbranched, saturated or unsaturated, and wherein the acyl residue and/or the alkyl residue can include at least one OH group, and
R², R³ and R⁴ each independently of one another signify
1) hydrogen or
2) an alkyl residue with 1 to 4 C atoms, which can be the same or different, saturated or unsaturated, and
3) a branched or unbranched hydroxyalkyl group with one to 4 carbon atoms with at least one and no more than three hydroxy groups e.g. —CH₂OH, —CH₂CH₂OH, —CHOHCHOH, —CH₂CHOHCH₃, —CH(CH₂OH)₂, —COH(CH₂OH)₂, —CH₂CHOHCH₂OH, —CH₂CH₂CH₂OH and hydroxybutyl residues, and
   A signifies an anion as described above and
   n signifies an integer between 1 and 10.

A composition in which the amine and/or the quaternized amine according to general formulae (Tkat3) is an amidoamine and/or a quaternized amidoamine, where R1 signifies a branched or unbranched, saturated or unsaturated acyl residue with 6 to 30 C atoms, which can include at least one OH group, is preferred. A fatty acid residue from oils and waxes is preferred here, in particular from natural oils and waxes. Lanolin, beeswax or candelilla wax are suitable examples of these.

Also preferred are those amidoamines and/or quaternized amidoamines, in which R2, R3 and/or R4 in the formula (Tkat3) signify a residue according to the general formula CH₂CH₂OR5, where R5 can have the meaning of alkyl residues with 1 to 4 carbon atoms, hydroxyethyl or hydrogen. The preferred size of n in the general formula (Tkat8) is an integer between 2 and 5.

The alkylamidoamines can either be present as such or can be converted to a quaternary compound in the composition by protonation in an appropriately acidic solution. The cationic alkylamidoamines are preferred according to the invention.

Examples of commercial products of this type according to the invention are Witcamine® 100, Incromine® BB, Mackine® 401 and other Mackine® grades, Adogen® S18V, and as permanent cationic amidoamines: Rewoquat® RTM 50, Empigen® CSC, Swanol® Lanoquat DES-50, Rewoquat® UTM 50, Schercoquat® BAS, Lexquat® AMG-BEO or Incroquat® Behenyl HE.

The above-mentioned cationic surfactants can be used individually or in any combinations with one another, wherein quantities between 0.01 and 10 wt. %, preferably in quantities of 0.01 to 7.5 wt. % and most particularly preferably in quantities of 0.1 to 5.0 wt. % are included. The best results of all are obtained here with quantities of 0.1 to 3.0 wt. %, based in each case on the overall composition of the respective agent.

The cationic and/or amphoteric polymers can be homopolymers or copolymers or polymers based on natural polymers, wherein the quaternary nitrogen groups are included either in the polymer chain or preferably as a substituent on one or more of the monomers. The monomers including ammonium groups can be copolymerized with non-cationic monomers. Suitable cationic monomers are unsaturated compounds capable of free-radical polymerization, which carry at least one cationic group, in particular ammonium-substituted vinyl monomers, such as e.g. trialkyl-methacryloxyalkylammonium, trialkylacryloxyalkylammonium, dialkyldiallylammonium and quaternary vinylammonium monomers with cyclic groups including cationic nitrogens, such as pyridinium, imidazolium or quaternary pyrrolidones, e.g. alkylvinylimidazolium, alkylvinylpyridinium, or alkylvinylpyrrolidone salts. The alkyl groups of these monomers are preferably lower alkyl groups, such as e.g. C₁ to C₇ alkyl groups, particularly preferably C₁ to C₃ alkyl groups.

The monomers including ammonium groups can be copolymerized with non-cationic monomers. Suitable comonomers are e.g. acrylamide, methacrylamide; alkyl and dialkyl acrylamide, alkyl and dialkyl methacrylamide, alkyl acrylate, alkyl methacrylate, vinyl caprolactone, vinyl caprolactam, vinyl pyrrolidone, vinyl esters, e.g. vinyl acetate, vinyl alcohol, propylene glycol or ethylene glycol, the alkyl groups of these monomers preferably being C₁ to C₇ alkyl groups, particularly preferably C₁ to C₃ alkyl groups.

A most preferred polymer is commercially available with the name Polyquaternium-74.

A particularly suitable homopolymer is the optionally crosslinked poly(methacryloyloxyethyl trimethylammonium chloride) with the INCI name Polyquaternium-37. Products of this type are commercially available, e.g. with the names Rheocare® CTH (Cosmetic Rheologies) and Synthalen® CR (3V Sigma).

The homopolymer is preferably used in the form of a non-aqueous polymer dispersion. Polymer dispersions of this type are commercially available with the names Salcare® SC 95 and Salcare® SC 96.

The following cationic polymers (a) are preferably used according to the invention in the agents according to the invention if the cationic polymers (a) fulfill one or more of the following features in relation to the above-mentioned formulae (I) to (IV):
R¹ and R⁴ each signify a methyl group,
X¹ denotes a group NH,
X² denotes a group NH,
A¹ and A² independently of one another denote ethane-1,2-diyl or propane-1,3-diyl,
R², R³, R⁵ and R⁶ independently of one another denote methyl or ethyl (particularly preferably methyl),
R⁷ denotes a (C₁₀ to C₂₄) alkyl group, in particular decyl (caprinyl), dodecyl (lauryl), tetradecyl (myristyl), hexadecyl (cetyl), octadecyl (stearyl), eicosyl (arachyl) or docosyl (behenyl).

It is preferred according to the invention to select the structural unit of formula (III) from at least one structural unit of formula (III-1) to (III-8)

[chemical structure]

[chemical structure]

Moreover, it has proved particularly preferred to select the structural unit according to formula (III-7) and/or of formula (III-8) as structural unit of formula (III). The structural unit of formula (III-8) is a most particularly preferred structural unit according to the invention.

Furthermore, with a view to achieving the object, it has proved to be preferred if the structural unit of formula (IV) is selected from at least one structural unit of formulae (IV-1) to (IV-8)

[chemical structure]

[chemical structure]

where R⁷ in each case denotes a (C₈ to C₃₀) alkyl group.

In turn, the structural units of formula (IV-7) and/or of formula (IV-8) where each R⁷ denotes octyl (capryl), decyl (caprinyl), dodecyl (lauryl), tetradecyl (myristyl), hexadecyl (cetyl), octadecyl (stearyl), eicosyl (arachyl) or docosyl (behenyl) are considered to be particularly preferred structural units of formula (IV). The structural unit of formula (IV-8) represents a most particularly preferred structural unit of formula (IV) according to the invention.

A cationic polymer that is most particularly preferably included in the agent according to the invention comprises at least one structural unit of formula (I), at least one structural unit of formula (II), at least one structural unit of formula (III-8) and at least one structural unit of formula (IV-8),

[chemical structure]

where R⁷ denotes octyl (capryl), decyl (caprinyl), dodecyl (lauryl), tetradecyl (myristyl), hexadecyl (cetyl), octadecyl (stearyl), eicosyl (arachyl) or docosyl (behenyl).

A most particularly preferred cationic polymer according to the invention is the copolymer of N-vinylpyrrolidone, N-vinylcaprolactam, N-(3-dimethylaminopropyl)methacrylamide and 3-(methacryloylamino)propyl-lauryl-dimethylammonium chloride (INCI name: Polyquaternium-69), which is marketed e.g. by ISP with the trade name AquaStyle® 300 (28-32 wt. % active substance in ethanol-water mixture, molecular weight 350000).

Suitable cationic polymers that are derived from natural polymers are cationic derivatives of polysaccharides, e.g. cationic derivatives of cellulose, starch or guar. Also suitable are chitosan and chitosan derivatives. Cationic polysaccharides have the general formula G-O-B-N+RₐR_{b}R_{c}A⁻

G is an anhydroglucose residue, e.g. starch or cellulose anhydroglucose;
B is a divalent compound group, e.g. alkylene, oxyalkylene, polyoxyalkylene or hydroxyalkylene;
Rₐ, R_{b} and R_{c}, independently of one another, are alkyl, aryl, alkylaryl, arylalkyl, alkoxyalkyl or alkoxyaryl with up to 18 C atoms in each case, the total number of C atoms in Rₐ, R_{b} and R_{c} preferably being a maximum of 20;
A⁻ is a conventional counteranion and is preferably chloride.

Cationic, i.e. quaternized celluloses are available on the market with different degrees of substitution, cationic charge density, nitrogen content and molecular weights. For example, Polyquaternium-67 is sold commercially with the names Polymer® SL or Polymer® SK (Amerchol). Another most preferred cellulose is sold with the trade name Mirustyle® CP by Croda. This is a trimonium and cocodimonium hydroxyethyl cellulose as a derivatized cellulose with the INCI name Polyquaternium-72. Polyquaternium-72 can be used both in solid form and already pre-dissolved in aqueous solution.

Other cationic celluloses are under the names Polymer JR® 400 (Amerchol, INCI name Polyquaternium-10) and Polymer Quatrisoft® LM-200 (Amerchol, INCI name Polyquaternium-24). Other commercial products are the compounds Celquat® H 100 and Celquat® L 200. Particularly preferred cationic celluloses are Polyquaternium-24, Polyquaternium-67 and Polyquaternium-72.

Suitable cationic guar derivatives are marketed with the trade name Jaguar® and have the INCI name Guar Hydroxypropyltrimonium Chloride. Furthermore, particularly suitable cationic guar derivatives are also marketed by Hercules with the name N-Hance®. Other cationic guar derivatives are sold by Cognis with the name Cosmedia®. A preferred cationic guar derivative is the commercial product AquaCat® from Hercules. This raw material is an already pre-dissolved cationic guar derivative. The cationic guar derivatives are preferred according to the invention.

A suitable chitosan is marketed e.g. by Kyowa Oil & Fat, Japan, with the trade name Flonac®. A preferred chitosan salt is chitosonium pyrrolidone carboxylate, which is marketed e.g. with the name Kytamer® PC by Amerchol, USA. Other chitosan derivatives are freely available on the market with the trade names Hydagen® CMF, Hydagen® HCMF and Chitolam® NB/101.

Other preferred cationic polymers are e.g.
cationized honey, e.g. the commercial product Honeyquat® 50,
polymeric dimethyldiallylammonium salts and their copolymers with esters and amides of acrylic acid and methacrylic acid. The products that are commercially available with the names Merquat® 100 (poly(dimethyldiallylammonium chloride)) and Merquat®550 (dimethyldiallylammonium chloride-acrylamide copolymer) are examples of these cationic polymers with the INCI name Polyquaternium-7,
vinylpyrrolidone-vinylimidazolium methochloride copolymers, as sold with the names Luviquat® FC 370, FC 550 and the INCI name Polyquaternium-16 as well as FC 905 and HM 552,
quaternized vinylpyrrolidone/dimethylaminoethyl methacrylate, for example vinylpyrrolidone/dimethylaminoethyl methacrylate methosulfate copolymer, which is sold by Gaf Co., USA, with the trade names Gafquat® 755 N and Gafquat® 734 and the INCI name Polyquaternium-11,
quaternized polyvinyl alcohol,
and the polymers known by the names Polyquaternium-2, Polyquaternium-17, Polyquaternium-18 and Polyquaternium-27 with quaternary nitrogen atoms in the polymer main chain,
   vinylpyrrolidone-vinylcaprolactam-acrylate terpolymers, as sold commercially with acrylic acid esters and acrylic acid amides as a third monomer building block, e.g. with the name Aquaflex® SF 40.

Particularly preferred amphoteric polymers are copolymers of at least one monomer (Mono1) or (Mono2) with the monomer (Mono3), in particular copolymers of the monomers (Mono2) and (Mono3). Most particularly preferably used amphoteric polymers according to the invention are copolymers of diallyldimethylammonium chloride and acrylic acid. These copolymers are marketed with the INCI name Polyquaternium-22, inter alia with the trade name Merquat® 280 (Nalco).

Most particularly preferably used amphoteric polymers according to the invention based on a comonomer (Mono4) are terpolymers of diallyldimethylammonium chloride, acrylamide and acrylic acid. These copolymers are marketed with the INCI name Polyquaternium-39, inter alia with the trade name Merquat® Plus 3330 (Nalco).

The amphoteric polymers can generally be used according to the invention either directly or in the form of a salt, which is obtained by neutralization of the polymers, e.g. with an alkali hydroxide.

The polymers described up to now represent only some of the polymers that can be used according to the invention. To avoid having to describe all cationic and/or amphoteric polymers that are suitable according to the invention, together with their composition, in summary the INCI declarations of the preferred polymers according to the invention are given. The preferred polymers according to the invention have the INCI name:

Polyquaternium-28, Polyquaternium-32, Polyquaternium-33, Polyquaternium-34, Polyquaternium-35, Polyquaternium-41, Polyquaternium-42, Polyquaternium-44, Polyquaternium-47, Polyquaternium-55, Polyquaternium-67, Polyquaternium-68, Polyquaternium-69, Polyquaternium-72, Polyquaternium-74, Polyquaternium-76, Polyquaternium-86, Polyquaternium-89 and Polyquaternium-95 and mixtures thereof.

The above-mentioned cationic polymers can be used individually or in any combinations with one another, with quantities of between 0.01 and 10 wt. %, preferably quantities of 0.01 to 7.5 wt. % and most particularly preferably in quantities of 0.1 to 5.0 wt. % being included. The best results of all are obtained here with quantities of 0.1 to 3.0 wt. %, based in each case on the overall composition of the respective agent.

In addition to the essential silicones described above, the compositions according to the invention can include other silicones. These optional silicones are preferably at least one silicone polymer selected from the group of the dimethiconols and/or the group of the amino functional silicones and/or the group of the dimethicones and/or the group of the cyclomethicones.

The dimethicones according to the invention can be both linear and branched, and cyclic or cyclic and branched. Linear dimethicones can be represented by the following structural formula (Si1):

(SiR¹₃)—O—(SiR²₂—O—)ₓ—(SiR¹₃) (Si1)

Branched dimethicones can be represented by the structural formula (Si1.1):

[chemical structure]

The residues R¹ and R², independently of one another, each denote hydrogen, a methyl residue, a C₂ to C₃₀ linear, saturated or unsaturated hydrocarbon residue, a phenyl residue and/or an aryl residue. The numbers x, y and z are integers and each independently of one another run from 0 to 50,000. The molecular weights of the dimethicones are between 1000 D and 10000000 D. The viscosities are between 100 and 10000000 cPs measured at 25° C. with the aid of a glass capillary viscometer in accordance with the Dow Corning corporate test method CTM 0004 of Jul. 20, 1970. Preferred viscosities are between 1000 and 5000000 cPs; most particularly preferred viscosities are between 10000 and 3000000 cPs. The most preferred range is between 50000 and 2000000 cPs. Most preferred are viscosities around the range of about 60,000 cPs. Reference may be made here, by way of example, to the product “Dow Corning 200 with 60000 cSt”.

Particularly preferred cosmetic or dermatological preparations according to the invention are characterized in that they include at least one silicone of the formula (Si1.2)

(CH₃)₃Si—[O—Si(CH₃)₂]ₓ—O—Si(CH₃)₃ (Si1.2),

in which x denotes a number from 0 to 100, preferably from 0 to 50, more preferably from 0 to 20 and in particular 0 to 10.

The dimethicones (Si1) are included in the compositions according to the invention in quantities of 0.01 to 10 wt. %, preferably 0.01 to 8 wt. %, particularly preferably 0.1 to 7.5 wt. % and in particular 0.1 to 5 wt. %, based on the overall composition.

Finally, the silicone compounds are understood to include the dimethiconols (Si8). The dimethiconols according to the invention can be both linear and branched and cyclic or cyclic and branched. Linear dimethiconols can be represented by the following structural formula (Si8-I):

(SiOHR¹₂)—O—(SiR²₂—O—)ₓ—(SiOHR¹₂) (Si8-I)

Branched dimethiconols can be represented by the structural formula (Si8-II):

[chemical structure]

The residues R¹ and R² independently of one another each denote hydrogen, a methyl residue, a C₂ to C₃₀ linear, saturated or unsaturated hydrocarbon residue, a phenyl residue and/or an aryl residue. The numbers x, y and z are integers and each independently of one another run from 0 to 50,000. The molecular weights of the dimethiconols are between 1000 D and 10000000 D. The viscosities are between 100 and 10000000 cPs measured at 25° C. with the aid of a glass capillary viscometer by the Dow Corning corporate test method CTM 0004 of Jul. 20, 1970. Preferred viscosities are between 1000 and 5000000 cPs; most particularly preferred viscosities are between 10000 and 3000000 cPs. The most preferred range is between 50000 and 2000000 cPs.

As examples of these products, the following commercial products are mentioned: Dow Corning 1-1254 Fluid, Dow Corning 2-9023 Fluid, Dow Corning 2-9026 Fluid, Abil OSW 5 (Degussa Care Specialties), Dow Corning 1401 Fluid, Dow Corning 1403 Fluid, Dow Corning 1501 Fluid, Dow Corning 1784 HVF Emulsion, Dow Corning 9546 Silicone Elastomer Blend, SM555, SM2725, SM2765, SM2785 (all four of the above GE Silicones), Wacker-Belsil CM 1000, Wacker-Belsil CM 3092, Wacker-Belsil CM 5040, Wacker-Belsil DM 3096, Wacker-Belsil DM 31 12 VP, Wacker-Belsil DM 8005 VP, Wacker-Belsil DM 60081 VP (all of the above Wacker-Chemie GmbH).

The dimethiconols (Si8) are in the compositions according to the invention in quantities of 0.01 to 10 wt. %, preferably 0.01 to 8 wt. %, particularly preferably 0.1 to 7.5 wt. % and in particular 0.1 to 5 wt. % of dimethiconol, based on the composition.

Particularly preferred agents according to the invention include one or more amino functional silicones. These silicones can be described e.g. by the formula (Si-2)

M(RₐQ_{b}SiO_{(4-a-b)/2})ₓ(R_{c}SiO_{(4-c)/2})_{y}M (Si-2)

wherein in the above formula
R is a hydrocarbon or a hydrocarbon residue with 1 to about 6 carbon atoms,
Q is a polar residue of the general formula —R¹HZ,
where
R¹ is a divalent linking group, which is bound to hydrogen and the residue Z, composed of carbon and hydrogen atoms, carbon, hydrogen and oxygen atoms or carbon, hydrogen and nitrogen atoms, and
Z is an organic, amino functional residue, which includes at least one amino functional group;
a assumes values in the range of about 0 to about 2,
b assumes values in the range of about 1 to about 3,
a+b is less than or equal to 3, and
c is a number in the range of about 1 to about 3, and
x is a number in the range of 1 to about 2000, preferably of about 3 to about 50 and most preferably of about 3 to about 25, and
y is a number in the range of about 20 to about 10,000, preferably of about 125 to about 10,000 and most preferably of about 150 to about 1000, and
M is a suitable silicone end group, as known in the prior art, preferably trimethylsiloxy.

Z according to formula (Si-2) is an organic, amino functional residue including at least one functional amino group. One possible formula for the said Z is NH(CH₂)_{z}NH₂, where z is an integer greater than or equal to 1. Another possible formula for the said Z is —NH(CH₂)_{z}(CH₂)_{zz}NH, where both z and zz independently of one another are an integer greater than or equal to 1, wherein this structure comprises diamino ring structures, such as piperazinyl. The said Z is most preferably an —NHCH₂CH₂NH₂ residue. Another possible formula for the said Z is —N(CH₂)_{z}(CH₂)_{zz}NX₂ or —NX₂, where each X of X₂ is selected independently from the group consisting of hydrogen and alkyl groups with 1 to 12 carbon atoms, and zz is 0.

Q according to formula (Si-2) is most preferably a polar amino functional residue of the formula

—CH₂CH₂CH₂NHCH₂CH₂NH₂.

In the formula (Si-2), a assumes values in the range of 0 to 2, b assumes values in the range of 2 to 3, a+b is less than or equal to 3 and c is a number in the range of 1 to 3. Suitable according to the invention are cationic silicone oils, such as e.g. the commercially available products Dow Corning (DC) 929 Emulsion, DC2-2078, DC5-71 13, SM-2059 (General Electric) and SLM-55067 (Wacker).

Particularly preferred agents according to the invention are characterized in that they include at least one amino functional silicone of the formula (Si3-a)

[chemical structure]

where m and n are numbers, the sum of which (m+n) is between 1 and 2000, preferably between 50 and 150, wherein n preferably assumes values of 0 to 1999 and in particular of 49 to 149, and m preferably assumes values of 1 to 2000, in particular of 1 to 10.

These silicones are known according to the INCI declaration as trimethylsilylamodimethicones and are available e.g. with the name Q2-7224 (manufacturer: Dow Corning; a stabilized trimethylsilylamodimethicone).

Particularly preferred are also agents according to the invention which include at least one amino functional silicone of the formula (Si-3b)

[chemical structure]

where
R denotes —OH, an (optionally ethoxylated and/or propoxylated) (C₁ to C₂₀) alkoxy group or a —CH₃ group,
R′ denotes —OH, a (C₁ to C₂₀) alkoxy group or a —CH₃ group and
m, n1 and n2 are numbers, the sum of which (m+n1+n2) is between 1 and 2000, preferably between 50 and 150, wherein the sum (n1+n2) preferably assumes values of 0 to 1999 and in particular of 49 to 149 and m preferably assumes values of 1 to 2000, in particular of 1 to 10.

These silicones are known according to the INCI Declaration as amodimethicones, or as functionalized amodimethicones, such as e.g. Bis(C13-15 Alkoxy) PG Amodimethicone (available e.g. as the commercial product: DC 8500 from Dow Corning), Trideceth-9 PG-Amodimethicone (available e.g. as the commercial product Silcare Silicone SEA from Clariant).

Suitable diquaternary silicones are selected from compounds of the general formula (Si3c)

[R¹R²R³N⁺-A-SiR⁷R⁸—(O—SiR⁹R¹⁰)ₙ—O—SiR¹¹R¹²-A-N⁺R⁴R⁵R⁶]2X⁻ (Si3c)

wherein the residues R1 to R6 independently of one another signify C1 to C22 alkyl residues, which can include hydroxy groups, and wherein preferably at least one of the residues has at least 8 C atoms and the remaining residues have 1 to 4 C atoms, the residues R7 to R12 independently of one another are the same or different and signify C1 to C10 alkyl or phenyl, A signifies a divalent organic compound group,
n is a number from 0 to 200, preferably from 10 to 120, particularly preferably from 10 to 40,
and X⁻ is an anion.

The divalent compound group is preferably a C1 to C12 alkylene or alkoxyalkylene group, which can be substituted with one or more hydroxyl groups. Particularly preferred is the group —(CH₂)₃—O—CH₂—CH(OH)—CH₂—.

The anion X⁻ can be a halide ion, an acetate, an organic carboxylate or a compound of the general formula RSO₃⁻, where R has the meaning of C1 to C4 alkyl residues.

A preferred diquaternary silicone has the general formula (Si3d)

[RN⁺Me₂-A-(SiMe₂O)ₙ—SiMe₂-A-N⁺Me₂R]2CH₃COO⁻ (Si3d),

wherein A is the group —(CH₂)₃—O—CH₂—CH(OH)—CH₂—,
R is an alkyl residue with at least 8 C atoms and n is a number from 10 to 120.

Suitable silicone polymers with two terminal, quaternary ammonium groups are known by the INCI name Quaternium-80. These are dimethylsiloxanes with two terminal trialkylammonium groups. Diquaternary polydimethylsiloxanes of this type are marketed by Evonik with the trade names Abil® Quat 3270, 3272 and 3474.

Preferred hair treatment agents according to the invention are characterized in that they include, based on their weight, 0.01 to 10 wt. %, preferably 0.01 to 8 wt. %, particularly preferably 0.1 to 7.5 wt. % and in particular 0.2 to 5 wt. % amino functional silicone(s) and/or diquaternary silicone.

Polyammonium-polysiloxane compounds are another silicone with amino functions according to the invention. The polyammonium-polysiloxane compounds can be obtained e.g. with the trade name Baysilone® from GE Bayer Silicones. The products with the names Baysilone TP 3911, SME 253 and SFE 839 are preferred here. Most particularly preferred is the use of Baysilone TP 3911 as active component in the compositions according to the invention. The polyammonium-polysiloxane compounds are used in the compositions according to the invention in a quantity of 0.01 to 10 wt. %, preferably 0.01 to 7.5, particularly preferably 0.01 to 5.0 wt. %, most particularly preferably of 0.05 to 2.5 wt. %, based in each case on the overall composition.

The cyclic dimethicones known under INCI as Cyclomethicones can also be used with preference according to the invention. In this case, cosmetic or dermatological preparations according to the invention are preferred which include at least one silicone of the formula (Si-4)

[chemical structure]

in which x denotes a number from 3 to 200, preferably from 3 to 10, more preferably from 3 to 7 and in particular 3, 4, 5 or 6.

Likewise, preferred agents according to the invention are characterized in that they include at least one silicone of the formula (Si-5)

R₃Si—[O—SiR₂]ₓ—(CH₂)ₙ—[O—SiR₂]_{y}—O—SiR₃ (Si-5),

in which R denotes the same or different residues from the group —H, -phenyl, -benzyl, —CH₂—CH(CH₃)Ph, the C₁₋₂₀ alkyl residues, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, x and y respectively denote a number from 0 to 200, preferably from 0 to 10, more preferably from 0 to 7 and in particular 0, 1, 2, 3, 4, 5 or 6, and n denotes a number from 0 to 10, preferably from 1 to 8 and in particular 2, 3, 4, 5, 6.

As additional silicones apart from the dimethicones, dimethiconols, amodimethicones and/or cyclomethicones according to the invention, water-soluble silicones can be included in the compositions according to the invention.

Corresponding hydrophilic silicones are selected e.g. from the compounds of the formulae (Si-6) and/or (Si-7). Particularly preferred water-soluble, silicone-based surfactants are selected from the group of the dimethicone copolyols, which are preferably alkoxylated, in particular polyethoxylated or polypropoxylated.

Dimethicone copolyols are preferably understood according to the invention to be polyoxyalkylene-modified dimethylpolysiloxanes of the general formulae (Si-6) or (Si-7):

[chemical structure]

where the residue R denotes a hydrogen atom, an alkyl group with 1 to 12 C atoms, an alkoxy group with 1 to 12 C atoms or a hydroxyl group, the residues R′ and R″ signify alkyl groups with 1 to 12 C atoms, x denotes an integer of 1 to 100, preferably of 20 to 30, y denotes an integer of 1 to 20, preferably of 2 to 10 and a and b denote integers of 0 to 50, preferably of 10 to 30.

Particularly preferred dimethicone copolyols within the meaning of the invention are e.g. the products that are marketed commercially with the trade name SILWET (Union Carbide Corporation) and DOW CORNING. Particularly preferred dimethicone copolyols are Dow Corning 190 and Dow Corning 193.

The dimethicone copolyols are in the compositions according to the invention in quantities of 0.01 to 10 wt. %, preferably 0.01 to 8 wt. %, particularly preferably 0.1 to 7.5 wt. % and in particular 0.1 to 5 wt. % of dimethicone copolyol, based on the composition.

Ester oils can be included with particular preference as oil bodies in active substance combination according to the invention. The ester oils are defined as follows:

Ester oils are to be understood as the esters of C₆-C₃₀ fatty acids with C₂-C₃₀ fatty alcohols. Preferred are the monoesters of fatty acids with alcohols having 2 to 24 C atoms. Examples of fatty acid parts that are used in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid as well as technical mixtures thereof. Examples of the fatty alcohol parts in the ester oils are isopropyl alcohol, caproyl alcohol, caprylyl alcohol, 2-ethylhexyl alcohol, capryl alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol as well as technical mixtures thereof. Particularly preferred according to the invention are isopropyl myristate (Rilanit® IPM), isononanoic acid C16-18-alkyl esters (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V).

Of course, the ester oils can also be alkoxylated with ethylene oxide, propylene oxide or mixtures of ethylene oxide and propylene oxide. The alkoxylation can be found in this case both on the fatty alcohol part and on the fatty acid part, and on both parts of the ester oils. It is preferred according to the invention, however, if the fatty alcohol was first alkoxylated and then esterified with fatty acid. In the formula (D4-II), these compounds are illustrated in general.

[chemical structure]

R1 here denotes a saturated or unsaturated, branched or unbranched, cyclic saturated cyclic unsaturated acyl residue with 6 to 30 carbon atoms,

AO denotes ethylene oxide, propylene oxide or butylene oxide,
X denotes a number between 1 and 200, preferably 1 and 100, particularly preferably between 1 and 50, more particularly preferably between 1 and 20, extremely preferably between 1 and 10 and most preferably between 1 and 5,
R2 denotes a saturated or unsaturated, branched or unbranched, cyclic saturated cyclic unsaturated alkyl, alkenyl, alkynyl, phenyl or benzyl residue with 6 to 30 carbon atoms. Examples of fatty acid parts used as residue R1 in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and technical mixtures thereof. Examples of the fatty alcohol parts as residue R2 in the ester oils are benzyl alcohol, isopropyl alcohol, caproyl alcohol, caprylyl alcohol, 2-ethylhexyl alcohol, capryl alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, linolyl alcohol, linoleyl alcohol, elaeostearyl alcohol, arachyl alcohol, gadoleyl alcohol, behenyl alcohol, erucyl alcohol and brassidyl alcohol as well as technical mixtures thereof. A particularly preferred ester oil according to the invention is available e.g. with the INCI name PPG-3 Benzyl Ether Myristate.

Ester oils are also to be understood to be:
dicarboxylic acid esters, such as di-n-butyl adipate, di-(2-ethylhexyl) adipate, di-(2-ethylhexyl) succinate and di-isotridecyl acetate as well as diol esters, such as ethylene glycol dioleate, ethylene glycol di-isotridecanoate, propylene glycol di(2-ethylhexanoate), propylene glycol diisostearate, propylene glycol dipelargonate, butanediol diisostearate, neopentyl glycol dicaprylate, as well as
symmetrical, asymmetrical or cyclic esters of carbonic acid with fatty alcohols, e.g. glycerol carbonate or dicaprylyl carbonate (Cetiol® CC),
trifatty acid esters of saturated and/or unsaturated, linear and/or branched fatty acids with glycerol,
fatty acid partial glycerides, which are monoglycerides, diglycerides and technical mixtures thereof. Typical examples are mono- and/or diglycerides based on caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid as well as technical mixtures thereof. Oleic acid monoglycerides are preferably used.

The ester oils are used in the agents according to the invention in a quantity of 0.01 to 20 wt. %, preferably 0.01 to 10.0 wt. %, particularly preferably 0.01 to 7.5 wt. %, extremely preferably 0.1 to 5.0 wt. %. It is, of course, also possible according to the invention to use several ester oils simultaneously.

Other oil bodies according to the invention are:
vegetable oils. Examples of these oils are sunflower oil, olive oil, soybean oil, rapeseed oil, almond oil, jojoba oil, orange oil, wheat germ oil, peach kernel oil and the liquid fractions of coconut oil. However, other triglyceride oils are also suitable, such as the liquid fractions of beef tallow and synthetic triglyceride oils.
liquid paraffin oils, isoparaffin oils and synthetic hydrocarbons as well as di-n-alkyl ethers with a total of between 12 and 36 C atoms, in particular 12 to 24 C atoms, such as e.g. di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether as well as di-tert-butyl ether, diisopentyl ether, di-3-ethyldecyl ether, tert.-butyl-n-octyl ether, isopentyl-n-octyl ether and 2-methylpentyl-n-octyl ether. The compounds 1,3-di-(2-ethyl-hexyl)cyclohexane (Cetiol® S) and di-n-octyl ether (Cetiol® OE), which are available as commercial products, can be preferred.

As natural oils, e.g. amaranth seed oil, apricot kernel oil, argan oil, avocado oil, babassu oil, cotton seed oil, borage seed oil, camelina oil, thistle oil, peanut oil, pomegranate seed oil, grapefruit seed oil, hemp oil, hazelnut oil, elderberry seed oil, blackcurrant seed oil, jojoba oil, cocoa butter, linseed oil, macadamia nut oil, corn oil, almond oil, marula oil, evening primrose oil, olive oil, palm oil, rapeseed oil, rice oil, sea buckthorn berry oil, sea buckthorn seed oil, sesame oil, shea butter, soybean oil, sunflower oil, grape seed oil, walnut oil or wild rose oil are used.

Of course, the hair treatment agents according to the invention also include, apart from the active substance combination according to the invention, additional constituents that are conventional in cosmetic compositions. The selection of these constituents generally depends on the intended use of the hair treatment agents. In the case of a shampoo, e.g. additional surface-active substances will be included. In the case of deep conditioners, optionally additional cationic compounds and additional care substances will be included.

In many cases, the agents include at least one surface-active substance, with anionic as well as zwitterionic, ampholytic, nonionic and cationic surface-active substances being suitable in principle. The selection of the surface-active substances depends on the nature of the agent.

Suitable as anionic surfactants (Tanion) in preparations according to the invention are all anionic surface-active substances that are suitable for use on the human body. Typical examples of anionic surfactants are:
linear and branched fatty acids with 8 to 30 C atoms (soaps),
ether carboxylic acids of the formula R—O—(CH₂—CH₂O)ₓ—CH₂—COOH, in which R is a linear alkyl group with 8 to 30 C atoms and x=0 or 1 to 16 and salts thereof,
acyl sarcosides with 8 to 24 C atoms in the acyl group,
acyl taurides with 8 to 24 C atoms in the acyl group,
acyl isethionates with 8 to 24 C atoms in the acyl group,
sulfosuccinic acid mono- and diallyl esters with 8 to 24 C atoms in the alkyl group and sulfosuccinic acid monoalkylpolyoxyethyl esters with 8 to 24 C atoms in the alkyl group and 1 to 6 oxyethyl groups.
linear alkane sulfonates with 8 to 24 C atoms,
linear alpha-olefin sulfonates with 8 to 24 C atoms,
alpha-sulfo fatty acid methyl esters of fatty acids with 8 to 30 C atoms,
alkyl sulfates and alkyl polyglycol ether sulfates of the formula R—O(CH₂—CH₂O)ₓ—OSO₃H, in which R is a preferably linear alkyl group with 8 to 30 C atoms and x=0 or 1 to 12,
hydroxysulfonates substantially corresponding to at least one of the following two formulae or mixtures thereof and salts thereof, CH₃—(CH₂)_{y}—CHOH—(CH₂)ₚ—(CH—SO₃M)-(CH₂)_{z}—CH₂—O—(CₙH₂ₙO)ₓ—H, and/or CH₃—(CH₂)_{y}—(CH—SO₃M)-(CH₂)ₚ—CHOH—(CH₂)_{z}—CH₂—O—(CnH₂ₙO)ₓ—H wherein in both of the formulae y and z=0 or integers from 1 to 18, p=0, 1 or 2 and the sum (y+z+p) is a number from 12 to 18, x=0 or a number from 1 to 30 and n is an integer from 2 to 4 and M=H or alkali, in particular sodium, potassium, lithium, alkaline earth, in particular magnesium, calcium, zinc and/or an ammonium ion, which may optionally be substituted, in particular mono-, di-, tri- or tetraammonium ions with C1 to C4 alkyl, alkenyl or aryl residues,
sulfated hydroxyalkyl polyethylene and/or hydroxyalkylene propylene glycol ethers of the formula R¹—(CHOSO₃M)-CHR³—(OCHR⁴—CH₂)ₙ—OR² with R¹ a linear alkyl residue with 1 to 24 C atoms, R² denotes a linear or branched, saturated alkyl residue with 1 to 24 C atoms, R³ denotes hydrogen or a linear alkyl residue with 1 to 24 C atoms, R⁴ denotes hydrogen or a methyl residue and M denotes hydrogen, ammonium, alkylammonium, alkanolammonium, where the alkyl and alkanol residues each have 1 to 4 C atoms, or a metal atom selected from lithium, sodium, potassium, calcium or magnesium and n denotes a number in the range of 0 to 12 and furthermore the total number of C atoms included in R¹ and R³ is 2 to 44,
sulfonates of unsaturated fatty acids with 8 to 24 C atoms and 1 to 6 double bonds,
esters of tartaric acid and citric acid with alcohols, which represent addition products of about 2-15 molecules of ethylene oxide and/or propylene oxide to fatty alcohols with 8 to 22 C atoms,
alkyl and/or alkenyl ether phosphates of the formula

R¹(OCH₂CH₂)ₙ—O—(PO—OX)—OR²,
in which R′ preferably denotes an aliphatic hydrocarbon residue with 8 to 30 carbon atoms, R² denotes hydrogen, a residue (CH₂CH₂O)ₙR² or X, n denotes numbers from 1 to 10 and X denotes hydrogen, an alkali or alkaline earth metal or NR³R⁴R⁵R⁶, with R³ to R⁶ independently of one another denoting hydrogen or a C₁ to C₄ hydrocarbon residue,
sulfated fatty acid alkylene glycol esters of the formula

RCO(AlkO)ₙSO₃M
in which RCO— denotes a linear or branched, aliphatic, saturated and/or unsaturated acyl residue with 6 to 22 C atoms, Alk denotes CH₂CH₂, CHCH₃CH₂ and/or CH₂CHCH₃, n denotes numbers from 0.5 to 5 and M denotes a metal, such as alkali metal, in particular sodium, potassium, lithium, alkaline earth metal, in particular magnesium, calcium, zinc, or ammonium ion, such as ⁺NR³R⁴R⁵R⁶, with R³ to R⁶ independently of one another denoting hydrogen or a C1 to C4 hydrocarbon residue,
monoglyceride sulfates and monoglyceride ether sulfates of the formula R⁸OC—(OCH₂CH₂)ₓ—OCH₂—[CHO(CH₂CH₂O)_{y}H]—CH₂O(CH₂CH₂O)_{z}—SO₃X, in which R⁸CO denotes a linear or branched acyl residue with 6 to 22 carbon atoms, x, y and z in total denote 0 or numbers from 1 to 30, preferably 2 to 10, and X denotes an alkali or alkaline earth metal. Typical examples of monoglyceride (ether) sulfates that are suitable within the meaning of the invention are the reaction products of lauric acid monoglyceride, coconut fatty acid monoglyceride, palmitic acid monoglyceride, stearic acid monoglyceride, oleic acid monoglyceride and tallow fatty acid monoglyceride and their ethylene oxide adducts with sulfur trioxide or chlorosulfonic acid in the form of their sodium salts. Monoglyceride sulfates are preferably used in which R⁸CO denotes a linear acyl residue with 8 to 18 carbon atoms,
amide ether carboxylic acids, R¹—CO—NR²—CH₂CH₂—O—(CH₂CH₂O)ₙCH₂COOM, with R¹ as a straight-chained or branched alkyl or alkenyl residue with a number of carbon atoms in the chain of 2 to 30, n denotes an integer of 1 to 20 and R² denotes hydrogen, a methyl, ethyl, propyl, isopropyl, n-butyl, t-butyl or isobutyl residue and M denotes hydrogen or a metal such as alkali metal, in particular sodium, potassium, lithium, alkaline earth metal, in particular magnesium, calcium, zinc, or an ammonium ion, such as ⁺NR³R⁴R⁵R⁶, with R³ to R⁶ independently of one another denoting hydrogen or a C1 to C4 hydrocarbon residue. Products of this type are available e.g. from Chem-Y with the product name Akypo®.
acylglutamates of the formula XOOC—CH₂CH₂CH(C(NH)OR)—COOX, in which RCO denotes a linear or branched acyl residue with 6 to 22 carbon atoms and 0 and/or 1, 2 or 3 double bonds and X denotes hydrogen, an alkali and/or alkaline earth metal, ammonium, alkylammonium, alkanolammonium or glucammonium,
condensation products of a water-soluble salt of a water-soluble protein hydrolyzate with a C8-C30 fatty acid. Products of this type have been commercially available for a long time with the trademarks Lamepon®, Maypon®, Gluadin®, Hostapon® KCG or Amisoft®.
alkyl and/or alkenyl oligoglycoside carboxylates, sulfates, phosphates and/or isethionates,
acyl lactylates and
hydroxy mixed ether sulfates.

Where the mild anionic surfactants include polyglycol ether chains, it is most particularly preferred that these have a narrow homolog distribution. Furthermore, in the case of mild anionic surfactants with polyglycol ether units, it is preferred for the number of glycol ether groups to be 1 to 20, preferably 2 to 15, particularly preferably 2 to 12. Particularly mild anionic surfactants with polyglycol ether groups without a restricted homolog distribution can also be obtained e.g. if on the one hand the number of polyglycol ether groups is 4 to 12 and Zn or Mg ions are selected as counterion. An example of this is the commercial product Texapon® ASV.

Nonionic surfactants (Tnio) are e.g.
addition products of 2 to 50 moles ethylene oxide and/or 0 to 5 moles propylene oxide to linear and branched fatty alcohols with 6 to 30 C atoms, the fatty alcohol polyglycol ethers or fatty alcohol polypropylene glycol ethers or mixed fatty alcohol polyethers,
addition products of 2 to 50 moles ethylene oxide and/or 0 to 5 moles propylene oxide to linear and branched fatty acids with 6 to 30 C atoms, the fatty acid polyglycol ethers or fatty acid polypropylene glycol ethers or mixed fatty acid polyethers,
addition products of 2 to 50 moles ethylene oxide and/or 0 to 5 moles propylene oxide to linear and branched alkylphenols with 8 to 15 C atoms in the alkyl group, the alkylphenol polyglycol ethers or the alkylpolypropylene glycol ethers, or mixed alkylphenol polyethers,
addition products of 2 to 50 moles ethylene oxide and/or 0 to 5 moles propylene oxide to linear and branched fatty alcohols with 8 to 30 C atoms, to fatty acids with 8 to 30 C atoms and to alkylphenols with 8 to 15 C atoms in the alkyl group, end-capped with a methyl or C₂-C₆ alkyl residue, such as e.g. the grades that are available with the trade names Dehydrol® LS, Dehydrol® LT (Cognis),
C₁₂-C₃₀ fatty acid mono- and diesters of addition products of 1 to 30 moles ethylene oxide to glycerol,
addition products of 5 to 60 moles ethylene oxide to castor oil and hydrogenated castor oil,
polyol fatty acid esters, such as e.g. the commercial product Hydagen® HSP (Cognis) or Sovermol® grades (Cognis),
alkoxylated triglycerides,
alkoxylated fatty acid alkyl esters of the formula (Tnio-1)

R¹CO—(OCH₂CHR²)_{w}OR³ (Tnio-1)
in which R¹CO denotes a linear or branched, saturated and/or unsaturated acyl residue with 6 to 22 carbon atoms, R² denotes hydrogen or methyl, R³ denotes linear or branched alkyl residues with 1 to 4 carbon atoms and w denotes numbers from 1 to 20,
amine oxides,
hydroxy mixed ethers, R¹O [CH₂CH(CH₃)O]ₓ(CH₂CHR²O)_{y}[CH₂CH(OH)R³]_{z} with R¹ denoting a linear or branched, saturated or unsaturated alkyl and/or alkenyl residue having 2 to 30 C atoms, R² denoting hydrogen, a methyl, ethyl, propyl or isopropyl residue, R³ denoting a linear or branched alkyl residue having 2 to 30 C atoms, x denoting 0 or a number from 1 to 20, Y denoting a number from 1 to 30 and z denoting the number 1, 2, 3, 4 or 5.
sorbitan fatty acid esters and addition products of ethylene oxide to sorbitan fatty acid esters, such as e.g. the polysorbates,
sugar fatty acid esters and addition products of ethylene oxide to sugar fatty acid esters,
addition products of ethylene oxide to fatty acid alkanolamides and fatty amines,
sugar surfactants of the alkyl and alkenyl oligoglycosides type,
sugar surfactants of the fatty acid N-alkylpolyhydroxyalkylamides type,
fatty acid amide polyglycol ethers, fatty amine polyglycol ethers,
mixed ethers or mixed formulas and polysorbates.

The surfactants (T) are used in quantities of 0.05-45 wt. %, preferably 0.1-30 wt. % and most particularly preferably of 0.5-25 wt. %, based on the total agent used according to the invention.

Emulsifiers that can be used according to the invention are e.g.
addition products of 4 to 30 moles ethylene oxide and/or 0 to 5 moles propylene oxide to linear fatty alcohols with 8 to 22 C atoms, to fatty acids with 12 to 22 C atoms and to alkylphenols with 8 to 15 C atoms in the alkyl group,
C₁₂-C₂₂ fatty acid mono- and diesters of addition products of 1 to 30 moles ethylene oxide to polyols with 3 to 6 carbon atoms, in particular to glycerol,
ethylene oxide and polyglycerol addition products to methyl glucoside fatty acid esters, fatty acid alkanolamides and fatty acid glucamides,
C₈-C₂₂ alkyl mono- and oligoglycosides and their ethoxylated analogs, wherein degrees of oligomerization of 1.1 to 5, in particular 1.2 to 2.0, and glucose as sugar component are preferred,
mixtures of alkyl (oligo)glucosides and fatty alcohols, for example the commercially available product Montanov®68,
addition products of 5 to 60 moles ethylene oxide to castor oil and hydrogenated castor oil,
partial esters of polyols having 3-6 carbon atoms with saturated fatty acids having 8 to 22 C atoms,
sterols, from both animal tissue (zoosterols, cholesterol, lanosterol) and vegetable fats (phytosterols, ergosterol, stigmasterol, sitosterol) or from fungi and yeasts (mycosterols),
phospholipids (lecithins, phosphatidylcholine),
fatty acid esters of sugars and sugar alcohols, such as sorbitol,
polyglycerols and polyglycerol derivatives, such as e.g. polyglycerol poly-12-hydroxystearate (commercial product Dehymuls® PGPH).

The agents according to the invention include the emulsifiers preferably in quantities of 0.1-25 wt. %, in particular 0.5-15 wt. %, based on the overall agent.

With particular preference, the compositions according to the invention include fatty substances (Fat) as a further active substance. Fatty substances (Fat) are to be understood as fatty acids, fatty alcohols, natural and synthetic waxes, which can be present both in solid form and as a liquid in aqueous dispersion, and natural and synthetic cosmetic oil components.

As fatty acids (Fatac), it is possible to use linear and/or branched, saturated and/or unsaturated fatty acids with 6-30 carbon atoms. Preferred are fatty acids with 10-22 carbon atoms. Among these, e.g. the isostearic acids, such as the commercial products Emersol® 871 and Emersol® 875, and isopalmitic acids, such as the commercial product Edenor® IP 95, as well as all other fatty acids marketed with the trade names Edenor® (Cognis) should be mentioned. Other typical examples of these fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and technical mixtures thereof. The fatty acid blends that are obtainable from coconut oil or palm oil are usually particularly preferred; the use of stearic acid is generally preferred in particular.

The quantity used here is 0.1-15 wt. %, based on the overall agent. Preferably the quantity is 0.5-10 wt. %, with quantities of 1-5 wt. % being most particularly advantageous.

As fatty alcohols (Fatal), it is possible to use saturated, mono- or polyunsaturated, branched or unbranched fatty alcohols with C₆-C₃₀, preferably C₁₀-C₂₂ and most particularly preferably C₁₂-C₂₂ carbon atoms. It is possible within the meaning of the invention to use e.g. decanol, octanol, octenol, dodecenol, decenol, octadienol, dodecadienol, decadienol, oleyl alcohol, erucyl alcohol, ricinoleyl alcohol, stearyl alcohol, isostearyl alcohol, cetyl alcohol, lauryl alcohol, myristyl alcohol, arachidyl alcohol, caprylyl alcohol, capryl alcohol, linoleyl alcohol, linolenyl alcohol and behenyl alcohol, as well as Guerbet alcohols thereof, this list being intended to be exemplary in nature and not limiting. However, the fatty alcohols preferably come from natural fatty acids, it generally being assumed that they are obtained from the esters of the fatty acids by reduction. It is likewise possible according to the invention to use those fatty alcohol blends that represent a mixture of different fatty alcohols. Substances of this type can be obtained commercially e.g. with the names Stenol®, e.g. Stenol® 1618 or Lanette®, e.g. Lanette® O or Lorol®, e.g. Lorol® C8, Lorol® C14, Lorol® C18, Lorol® C8-18, HD-Ocenol®, Crodacol®, e.g. Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. It is, of course, also possible according to the invention to use wool wax alcohols, as can be obtained commercially e.g. with the names Corona®, White Swan®, Coronet® or Fluilan®. The fatty alcohols are used in quantities of 0.1-30 wt. %, based on the overall preparation, preferably in quantities of 0.1-20 wt. %.

As natural or synthetic waxes (Fatwax), it is possible according to the invention to use solid paraffins or isoparaffins, carnauba waxes, beeswaxes, candelilla waxes, ozokerite, ceresin, cetaceum, sunflower wax, fruit waxes, such as e.g. apple wax or citrus wax, or microwaxes of PE or PP. Waxes of this type are available e.g. through Kahl & Co., Trittau.

The quantity used is 0.1-50 wt. %, based on the overall agent, preferably 0.1-20 wt. % and particularly preferably 0.1-15 wt. %, based on the overall agent.

The total quantity of oil and fat components in the agents according to the invention is generally 0.5-75 wt. %, based on the overall agent. Quantities of 0.5-35 wt. % are preferred according to the invention.

Another synergistic active substance according to the invention in the compositions according to the invention with the active substance complex according to the invention is protein hydrolyzates and/or derivatives thereof (P).

According to the invention, protein hydrolyzates of vegetable as well as animal or marine or synthetic origin can be used.

Animal protein hydrolyzates are e.g. elastin, collagen, keratin, silk and milk protein hydrolyzates, which can also be present in the form of salts. Products of this type are marketed e.g. with the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

Furthermore, preferred vegetable protein hydrolyzates according to the invention are e.g. soybean, almond, pea, moringa, potato and wheat protein hydrolyzates. Products of this type are available e.g. with the trademarks Gluadin® (Cognis), DiaMin® (Diamalt), Lexein® (Inolex), Hydrosoy® (Croda), Hydrolupin® (Croda), Hydrosesame® (Croda), Hydrotritium® (Croda), Crotein® (Croda) and Puricare® LS 9658 from Laboratoires Sérobiologiques.

Other preferred protein hydrolyzates according to the invention are of marine origin. These include e.g. collagen hydrolyzates from fish or algae as well as protein hydrolyzates from mussels or pearl hydrolyzates. Examples of pearl extracts according to the invention are the commercial products Pearl Protein Extract BG® or Crodarom® Pearl.

Furthermore, the protein hydrolyzates and derivatives thereof include cationized protein hydrolyzates, with the basic protein hydrolyzate originating from animals, e.g. from collagen, milk or keratin, from plants, e.g. from wheat, maize, rice, potatoes, soybean or almonds, from marine life forms, e.g. from fish collagen or algae, or protein hydrolyzates obtained by biotechnology. As typical examples of the cationic protein hydrolyzates and derivatives according to the invention, the products that are mentioned under the INCI names in the “International Cosmetic Ingredient Dictionary and Handbook”, (seventh edition 1997, The Cosmetic, Toiletry and Fragrance Association 1101 17th Street, N.W., Suite 300, Washington, D.C. 20036-4702) and that are commercially available should be mentioned.

The protein hydrolyzates (P) are included in the compositions in concentrations of 0.001 wt. % up to 20 wt. %, preferably of 0.05 wt. % up to 15 wt. % and most particularly preferably in quantities of 0.05 wt. % up to 5 wt. %.

Another preferred group of ingredients in the compositions according to the invention with the active substance complex according to the invention are vitamins, provitamins or vitamin precursors.

Vitamins, pro-vitamins and vitamin precursors belonging to the groups A, B, C, E, F and H are particularly preferred here.

The group of substances referred to as vitamin A includes retinol (vitamin A₁) and 3,4-didehydroretinol (vitamin A₂). β-Carotene is the provitamin of retinol. Suitable according to the invention as vitamin A component are e.g. vitamin A acid and esters thereof, vitamin A aldehyde and vitamin A alcohol and esters thereof, such as the palmitate and the acetate. The agents according to the invention include the vitamin A component preferably in quantities of 0.05-1 wt. %, based on the overall preparation.

The vitamin B group or vitamin B complex includes, inter alia:

Vitamin B₁ (thiamine)

Vitamin B₂ (riboflavin)

Vitamin B₃. This name often covers the compounds nicotinic acid and nicotinamide (niacinamide). Preferred according to the invention is nicotinamide, which is included in the agents used according to the invention preferably in quantities of 0.05 to 1 wt. %, based on the overall agent.

Vitamin B₅ (pantothenic acid, panthenol and pantolactone). Within the context of this group, preferably panthenol and/or pantolactone is used. Derivatives of panthenol that can be used according to the invention are in particular the esters and ethers of panthenol and cationically derivatized panthenols. Individual representatives are e.g. panthenol triacetate, panthenol monoethyl ether and monoacetate thereof as well as cationic panthenol derivatives. Pantothenic acid is preferably used as a derivative in the form of the more stable calcium salts and sodium salts (Ca pantothenate, Na pantothenate) in the present invention.

Vitamin B6 (pyridoxine as well as pyridoxamine and pyridoxal).

The above compounds of the vitamin B type, in particular vitamin B₃, B₅ and B₆, are included in the agents according to the invention preferably in quantities of 0.05-10 wt. %, based on the overall agent. Quantities of 0.1-5 wt. % are particularly preferred.

Vitamin C (ascorbic acid). Vitamin C is used in the agents according to the invention preferably in quantities of 0.1 to 3 wt. %, based on the overall agent. Use in the form of the palmitic acid ester, glucosides or phosphates can be preferred. Use in combination with tocopherols can likewise be preferred.

Vitamin E (tocopherols, in particular α-tocopherol). Tocopherol and derivatives thereof, including in particular the esters, such as the acetate, the nicotinate, the phosphate and the succinate, are included in the agents according to the invention preferably in quantities of 0.05-1 wt. %, based on the overall agent.

Vitamin F. The term “vitamin F” is generally understood to mean essential fatty acids, in particular linoleic acid, linolenic acid and arachidonic acid.

Vitamin H. The compound (3aS,4S,6aR)-2-oxohexahydrothieno[3,4-d]-imidazole-4-valeric acid is referred to as vitamin H, but its trivial name biotin has now become accepted. Biotin is included in the agents according to the invention preferably in quantities of 0.0001 to 1.0 wt. %, in particular in quantities of 0.001 to 0.01 wt. %.

The compositions according to the invention preferably include vitamins, provitamins and vitamin precursors from the groups A, B, E and H. Panthenol, pantolactone, pyridoxine and derivatives thereof as well as nicotinamide and biotin are particularly preferred.

A particularly preferred group of ingredients in the cosmetic compositions according to the invention are the betaines mentioned below: carnitine, carnitine tartrate, carnitine magnesium citrate, acetyl carnitine, betalains, 1,1-dimethylproline, choline, choline chloride, choline bitartrate, choline dihydrogen citrate and the compound N,N,N-trimethylglycine, which is referred to in the literature as betaine.

In another preferred embodiment according to the invention, the compositions according to the invention include bioquinones. In the agents according to the invention, suitable bioquinones are understood to be one or more ubiquinone(s) and/or plastoquinone(s). The preferred ubiquinones according to the invention have the following formula:

[chemical structure]

The coenzyme Q-10 is most preferred here.

Preferred compositions according to the invention include purine and/or purine derivatives in relatively narrow quantitative ranges. Preferred cosmetic agents according to the invention here are characterized in that they include—based on their weight—0.001 to 2.5 wt. %, preferably 0.0025 to 1 wt. %, particularly preferably 0.005 to 0.5 wt. % and in particular 0.01 to 0.1 wt. % purine(s) and/or purine derivative(s). Preferred cosmetic agents according to the invention are characterized in that [they] include purine, adenine, guanine, uric acid, hypoxanthine, 6-purinethiol, 6-thioguanine, xanthine, caffeine, theobromine or theophylline. In hair cosmetic preparations, caffeine is most preferred.

In another preferred embodiment of the present invention, the cosmetic agent includes ectoine ((S)-2-methyl-1,4,5,6-tetrahydro-4-pyrimidine carboxylic acid).

Particularly preferred are agents which include—based on their weight—0.00001 to 10.0 wt. %, preferably 0.0001 to 5.0 wt. % and in particular 0.001 to 3 wt. % of the active substances from the group that is made up of carnitine, coenzyme Q-10, ectoine, a vitamin from the B series, a purine and derivatives or physiologically acceptable salts thereof.

A most particularly preferred care additive in the hair treatment agents according to the invention is taurine. Taurine is understood to be exclusively 2-aminoethanesulfonic acid and a derivative to be the explicitly mentioned derivatives of taurine. The derivatives of taurine are understood to be N-monomethyltaurine, N,N-dimethyltaurine, taurine lysylate, taurine tartrate, taurine ornithate, lysyl taurine and ornithyl taurine.

Particularly preferred are agents according to the invention which include—based on their weight—0.0001 to 10.0 wt. %, preferably 0.0005 to 5.0 wt. %, particularly preferably 0.001 to 2.0 wt. % and in particular 0.001 to 1.0 wt. % taurine and/or a derivative of taurine.

The action of the compositions according to the invention can furthermore be increased by a 2-pyrrolidinone-5-carboxylic acid and derivatives thereof (J). Preferred are the sodium, potassium, calcium, magnesium or ammonium salts, in which the ammonium ion carries one to three C₁ to C₄ alkyl groups in addition to hydrogen. The sodium salt is most particularly preferred. The quantities used in the agents according to the invention are 0.05 to 10 wt. %, based on the overall agent, particularly preferably 0.1 to 5 and in particular 0.1 to 3 wt. %.

Through the use of plant extracts as care substances, the hair treatment agents according to the invention can be formulated to be particularly close to nature and yet very effective in their care performance. It may even be possible in this case to omit otherwise conventional preservatives. According to the invention, in particular the extracts of green tea, oak bark, nettles, hamamelis, hops, henna, chamomile, burdock root, horsetail, hawthorn, lime blossom, almond, aloe vera, fir needle, horse chestnut, sandalwood, juniper, coconut, mango, apricot, lime, wheat, kiwi, melon, orange, grapefruit, sage, rosemary, birch, mallow, valerian, lady's smock, wild thyme, yarrow, thyme, melissa, rest harrow, coltsfoot, marshmallow, meristem, ginseng, coffee, cocoa, moringa, ginger root and ayurvedic plant extracts, such as e.g. Aegle marmelos (Bilva), Cyperus rotundus (Nagarmotha), Emblica officinalis (Amalaki), Morida citrifolia (Ashyuka), Tinospora cordifolia (Guduchi), Santalum album (Chandana), Crocus sativus (Kumkuma), Cinnamonum zeylanicum and Nelumbo nucifera (Kamala), graminaceous plants, such as wheat, barley, rye, oats, spelt, maize, the various types of millet (proso millet, finger millet, foxtail millet as examples), sugarcane, perennial ryegrass, meadow foxtail, tall oatgrass, bentgrass, meadow fescue, purple moor grass, bamboo, cottongrass, pennisetums, Andropogonodeae (Imperata cylindrica, also known as blady grass or cogon grass), buffalo grass, cord-grasses, dog's tooth grasses, lovegrasses, Cymbopogon (lemongrass), Oryzeae (rice), Zizania (wild rice), beach grass, blue oat grass, soft grasses, quaking grasses, meadow grasses, wheatgrasses and echinacea, in particular Echinacea purpurea (L.) Moench, all types of vine and pericarp of Litchi chinensis are preferred.

The plant extracts can be used according to the invention both in pure form and in dilute form. Where they are used in dilute form, they usually include approx. 2-80 wt. % active substance and, as solvent, the extracting agent or mixture of extracting agents used to obtain them.

It may occasionally be necessary to use anionic polymers. Examples of anionic monomers from which these polymers can consist are acrylic acid, methacrylic acid, crotonic acid, maleic anhydride and 2-acrylamido-2-methylpropanesulfonic acid. The acid groups here can be completely or partially present as a sodium, potassium, ammonium, mono- or triethanolammonium salt. Preferred monomers are 2-acrylamido-2-methylpropanesulfonic acid and acrylic acid.

Anionic polymers including 2-acrylamido-2-methylpropanesulfonic acid as sole monomer or comonomer, wherein the sulfonic acid group can be completely or partially present as a sodium, potassium, ammonium, mono- or triethanolammonium salt, have proved most particularly effective.

Particularly preferred is the homopolymer of 2-acrylamido-2-methylpropanesulfonic acid, which is commercially available e.g. with the name Rheothik®11-80.

Preferred non-ionogenic monomers are acrylamide, methacrylamide, acrylic acid ester, methacrylic acid ester, vinylpyrrolidone, vinyl ether and vinyl ester.

Preferred anionic copolymers are acrylic acid-acrylamide copolymers and in particular polyacrylamide copolymers with monomers that include sulfonic acid groups. A polymer of this type is included in the commercial product Sepiger®305 from SEPPIC.

Likewise preferred anionic homopolymers are uncrosslinked and crosslinked polyacrylic acids. Preferred crosslinking agents in this case can be allyl ethers of pentaerythritol, of sucrose and of propylene. Compounds of this type are commercially available e.g. with the trademark Carbopol®.

Copolymers of maleic anhydride and methyl vinyl ether, in particular those with crosslinkages, are also color-retaining polymers. A maleic acid-methyl vinyl ether copolymer crosslinked with 1,9-decadiene is commercially available with the name Stabileze® QM.

The anionic polymers are included in the agents according to the invention preferably in quantities of 0.05 to 10 wt. %, based on the overall agent. Quantities of 0.1 to 5 wt. % are particularly preferred.

In a further embodiment, the agents according to the invention can include non-ionogenic polymers.

Suitable non-ionogenic polymers are e.g.:
Vinylpyrrolidone/vinyl ester copolymers, as marketed e.g. with the trademark Luviskol® (BASF). Luviskol® VA 64 and Luviskol® VA 73, both of them vinylpyrrolidone/vinyl acetate copolymers, are likewise preferred nonionic polymers.
Cellulose ethers, such as hydroxypropyl cellulose, hydroxyethyl cellulose and methyl hydroxypropyl cellulose, as marketed e.g. with the trademarks Culminal® and Benecel® (AQUALON) and Natrosol® grades (Hercules).
Starch and derivatives thereof, in particular starch ethers, e.g. Structure® XL (National Starch), a multifunctional, salt-tolerant starch;
Shellac
Polyvinylpyrrolidones, as marketed e.g. with the name Luviskol® (BASF).

The nonionic polymers are included in the compositions according to the invention preferably in quantities of 0.05 to 10 wt. %, based on the overall agent. Quantities of 0.1 to 5 wt. % are particularly preferred.

In another embodiment, the agents according to the invention should additionally include at least one UV light protective filter. UVB filters can be oil-soluble or water-soluble.

As oil-soluble substances, e.g. the following should be mentioned:
3-benzylidene camphor, e.g. 3-(4-methylbenzylidene) camphor;
4-aminobenzoic acid derivatives, preferably 4-(dimethylamino)benzoic acid 2-ethylhexyl ester, 4-(dimethylamino)benzoic acid 2-octyl ester and 4-(dimethylamino)benzoic acid amyl ester;
esters of cinnamic acid, preferably 4-methoxycinnamic acid 2-ethylhexyl ester, 4-methoxycinnamic acid propyl ester, 4-methoxycinnamic acid isoamyl ester, 2-cyano-3-phenylcinnamic acid 2-ethylhexyl ester (octocrylene);
esters of salicylic acid, preferably salicylic acid 2-ethylhexyl ester, salicylic acid 4-isopropylbenzyl ester, salicylic acid homomethyl ester;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably 4-methoxybenzmalonic acid di-2-ethylhexyl ester;
triazine derivatives, such as e.g. 2,4,6-trianilino-(p-carbo-2′-ethyl-1′-hexyloxy)-1,3,5-triazine and octyltriazone.
propane-1,3-diones, such as e.g. 1-(4-tert.butylphenyl)-3-(4′-methoxyphenyl)propane-1,3-dione.

The following are suitable as water-soluble substances:
2-phenylbenzimidazole-5-sulfonic acid and alkali, alkaline earth, ammonium, alkylammonium, alkanolammonium and glucammonium salts thereof;
sulfonic acid derivatives of benzophenones, preferably 2-hydroxy-4-methoxybenzophenone-5-sulfonic acid and salts thereof;
sulfonic acid derivatives of 3-benzylidene camphor, such as e.g. 4-(2-oxo-3-bornylidenemethyl)benzenesulfonic acid and 2-methyl-5-(2-oxo-3-bornylidene)sulfonic acid and salts thereof.

As typical UV-A filters, derivatives of benzoyl methane are particularly suitable, such as e.g. 1-(4′-tert.butylphenyl)-3-(4′-methoxyphenyl)propane-1,3-dione or 1-phenyl-3-(4′-isopropylphenyl)propane-1,3-dione. The UV-A and UV-B filters can, of course, also be used in mixtures. In addition to the aforementioned soluble substances, insoluble pigments are also suitable for this purpose, in particular finely dispersed metal oxides or salts, such as e.g. titanium dioxide, zinc oxide, iron oxide, aluminum oxide, cerium oxide, zirconium oxide, silicates (talc), barium sulfate and zinc stearate. The particles in this case should have an average diameter of less than 100 nm, preferably between 5 and 50 nm and in particular between 15 and 30 nm. They can have a spherical shape, but those particles that possess an ellipsoid shape or a shape deviating from the spherical form in another way can also be used.

Furthermore, the cosmetic agents can include additional active substance, auxiliary substances and additives, such as e.g.
structurants, such as maleic acid and lactic acid,
swelling agents, such as urea, allantoin, carbonates or hydantoin,
dimethyl isosorbide and cyclodextrins,
dyes for coloring the agent,
active anti-dandruff substances, such as piroctone olamine, zinc omadine and climbazole,
complexing agents, such as EDTA, NTA, β-alanine diacetic acid and phosphonic acids,
opacifiers, such as latex, styrene/PVP and styrene/acrylamide copolymers,
pearlescent agents, such as ethylene glycol mono- and distearate and PEG-3 distearate,
pigments,
stabilizers for hydrogen peroxide and other oxidizing agents,
propellants, such as propane-butane mixtures, N₂O, dimethyl ether, CO₂ and air,
antioxidants,
perfume oils, fragrances and odorants.

With regard to further optional components and the amounts of these components used, explicit reference is made to the relevant handbooks known to the person skilled in the art.

The invention therefore also provides a method for hair treatment, in which a hair treatment agent according to claim 1 is applied onto the hair and rinsed off the hair after a period of exposure.

The period of exposure is preferably a few seconds to 100 minutes, particularly preferably 1 to 50 minutes and most particularly preferably 1 to 30 minutes.

A method in which a cosmetic agent according to claim 1 is applied onto the hair and remains there is also according to the invention. “Remains on the hair” is understood according to the invention to mean that the agent is not rinsed out of the hair immediately after its application. Instead, in this case the agent remains on the hair for more than 100 minutes up to the next hair wash.

Finally, the use of a composition as described above to reduce and/or delay flaking of the scalp is according to the invention.

The following examples are intended to explain the subject-matter of the present invention but without limiting it.

### EXAMPLES

Unless otherwise specified, all quantitative data are parts by weight. The following formulations were prepared using known methods of production.

Care spray, also suitable for application as a foam and/or deep conditioner:

| | K1 | K2 | K3 | K4 | K5 | K6 | K7 | K8 | K9 | K10 | K11 |
|---|---|---|---|---|---|---|---|---|---|---|---|
| Polymer JR 400 | 0.2 | 0.2 | 0.2 | 0.2 | 0.2 | 0.2 | 0.2 | 0.2 | 0.2 | 0.2 | 0.2 |
| Armocare VGH 70 | 0.5 | 0.5 | 0.5 | 0.5 | 0.5 | 0.5 | 0.5 | 0.5 | 0.5 | 0.5 | 0.5 |
| Stearamidopropyl | 0.5 | 0.5 | 0.5 | 0.5 | 0.5 | 0.5 | 0.5 | 0.5 | 0.5 | 0.5 | 0.5 |
| Dimethylamine | | | | | | | | | | | |
| PVP/VA | 1.0 | 1.0 | 1.0 | 1.0 | 1.0 | 1.0 | 1.0 | 1.0 | 1.0 | 1.0 | 1.0 |
| copolymer 60/40 | | | | | | | | | | | |
| Polyquaternium-71 | 0.5 | 0.5 | 0.5 | 0.5 | 0.5 | 0.5 | 0.5 | 0.5 | 0.5 | 0.5 | 0.5 |
| Polyquaternium-69 | — | 0.5 | — | — | — | — | — | — | — | 0.5 | — |
| Polyquaternium-39 | — | — | 0.5 | — | — | — | — | — | — | — | — |
| Polyquaternium-72 | — | — | — | 0.5 | — | — | — | — | — | 0.3 | 0.3 |
| Polyquaternium-16 | — | — | — | — | 0.5 | — | — | — | — | — | — |
| Polyquaternium-55 | — | — | — | — | — | 0.5 | — | — | — | — | — |
| Polyquaternium-44 | — | — | — | — | — | — | 0.5 | — | — | — | — |
| Polyquaternium-68 | — | — | — | — | — | — | — | 0.5 | — | — | — |
| Polyquaternium-89 | — | — | — | — | — | — | — | — | 0.5 | — | 0.3 |
| Panthenol | 0.2 | 0.2 | 0.2 | 0.2 | 0.2 | 0.2 | 0.2 | 0.2 | 0.2 | 0.2 | 0.2 |
| Cetrimonium | 5.0 | 5.0 | 5.0 | 5.0 | 5.0 | 5.0 | 5.0 | 5.0 | 5.0 | 5.0 | 5.0 |
| chloride | | | | | | | | | | | |
| Ceteareth-25 | 0.3 | 0.3 | 0.3 | 0.3 | 0.3 | 0.3 | 0.3 | 0.3 | 0.3 | 0.3 | 0.3 |
| Protein hydrolyzate | 0.2 | 0.2 | 0.2 | 0.2 | 0.2 | 0.2 | 0.2 | 0.2 | 0.2 | 0.2 | 0.2 |
| Dow Corning 193 | 0.2 | 0.2 | 0.2 | 0.2 | 0.2 | 0.2 | 0.2 | 0.2 | 0.2 | 0.2 | 0.2 |
| Silicone | 0.5 | 0.5 | 0.5 | 0.5 | 0.5 | 0.5 | 0.5 | 0.5 | 0.5 | 0.5 | 0.5 |
| Quaternium-22 | | | | | | | | | | | |
| Coco Betaine | 0.5 | 0.5 | 0.5 | 0.5 | 0.5 | 0.5 | 0.5 | 0.5 | 0.5 | 0.5 | 0.5 |
| Water, preservative | to 100 | to 100 | to 100 | to 100 | to 100 | to 100 | to 100 | to 100 | to 100 | to 100 | to 100 |
| and optionally | | | | | | | | | | | |
| perfume oils | | | | | | | | | | | |

The pH values of all of the formulations were adjusted to 2 to 6.

For application as a foam, the relevant formulation is either packaged in an aerosol container with a propellant or delivered as a foam from a pump bottle with an appropriate pump attachment, such as e.g. an Airfoamer.

For use as a deep conditioner or cream, fatty alcohol such as cetyl stearyl alcohol and/or ethylene glycol distearate and/or glycerol monostearate is added to the above formulations in quantities of 0.2 to 5.0 wt. %.

Shampoo:

| | S1 | S2 | S3 | S4 | S5 | S6 | S7 | S8 | S9 | S10 | S11 |
|---|---|---|---|---|---|---|---|---|---|---|---|
| Texapon ® N70 | 15.0 | 15.0 | 15.0 | 15.0 | 15.0 | 15.0 | 15.0 | 15.0 | 15.0 | 15.0 | 15.0 |
| Arlypon ® F | 0.15 | 0.15 | 0.15 | 0.15 | 0.15 | 0.15 | 0.15 | 0.15 | 0.15 | 0.15 | 0.15 |
| Antil ® 141 | 0.15 | 0.15 | 0.15 | 0.15 | 0.15 | 0.15 | 0.15 | 0.15 | 0.15 | 0.15 | 0.15 |
| Disodium | 6.0 | 6.0 | 6.0 | 6.0 | 6.0 | 6.0 | 6.0 | 6.0 | 6.0 | 6.0 | 6.0 |
| Cocoampho- | | | | | | | | | | | |
| diacetate | | | | | | | | | | | |
| Polyquaternium- | 0.5 | 0.5 | 0.5 | 0.5 | 0.5 | 0.5 | 0.5 | 0.5 | 0.5 | 0.5 | 0.5 |
| 71 | | | | | | | | | | | |
| Polyquaternium- | — | 0.5 | — | — | — | — | — | — | — | 0.5 | — |
| 69 | | | | | | | | | | | |
| Polyquaternium- | — | — | 0.5 | — | — | — | — | — | — | — | — |
| 39 | | | | | | | | | | | |
| Polyquaternium- | — | — | — | 0.5 | — | — | — | — | — | 0.3 | 0.3 |
| 72 | | | | | | | | | | | |
| Polyquaternium- | — | — | — | — | 0.5 | — | — | — | — | — | — |
| 16 | | | | | | | | | | | |
| Polyquaternium- | — | — | — | — | — | 0.5 | — | — | — | — | — |
| 55 | | | | | | | | | | | |
| Polyquaternium- | — | — | — | — | — | — | 0.5 | — | — | — | — |
| 44 | | | | | | | | | | | |
| Polyquaternium- | — | — | — | — | — | — | — | 0.5 | — | — | — |
| 68 | | | | | | | | | | | |
| Polyquaternium- | — | — | — | — | — | — | — | — | 0.5 | — | 0.3 |
| 89 | | | | | | | | | | | |
| Cetiol ® HE | 3.0 | 3.0 | 3.0 | 3.0 | 3.0 | 3.0 | 3.0 | 3.0 | 3.0 | 3.0 | 3.0 |
| Panthenol | 0.2 | 0.2 | 0.2 | 0.2 | 0.2 | 0.2 | 0.2 | 0.2 | 0.2 | 0.2 | 0.2 |
| Dow Corning ® | 0.1 | 0.1 | 0.1 | 0.1 | 0.1 | 0.1 | 0.1 | 0.1 | 0.1 | 0.1 | 0.1 |
| 193 | | | | | | | | | | | |
| Silicone | 1.0 | 1.0 | 1.0 | 1.0 | 1.0 | 1.0 | 1.0 | 1.0 | 1.0 | 1.0 | 1.0 |
| Quaternium-22 | | | | | | | | | | | |
| Protein | 0.5 | 0.5 | 0.5 | 0.5 | 0.5 | 0.5 | 0.5 | 0.5 | 0.5 | 0.5 | 0.5 |
| hydrolyzate | | | | | | | | | | | |
| Cremophor ® | 0.5 | 0.5 | 0.5 | 0.5 | 0.5 | 0.5 | 0.5 | 0.5 | 0.5 | 0.5 | 0.5 |
| HRE 60 | | | | | | | | | | | |
| Water, | to 100 | to 100 | to 100 | to 100 | to 100 | to 100 | to 100 | to 100 | to 100 | to 100 | to 100 |
| preservative and | | | | | | | | | | | |
| optionally | | | | | | | | | | | |
| perfume oils | | | | | | | | | | | |

The pH values of all of the formulations were adjusted to 4.5 to 5.8.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A hair treatment agent comprising, in a suitable cosmetic carrier—based in each case on the overall composition of the agent—
a) at least 0.01 to 10.0 wt. % Polyquaternium-71 and
b) at least one cationic amino silicone having at least three terminal amino functional groups in a total quantity of 0.01 to 5.0 wt. %.

2. The hair treatment agent according to claim 1, further comprising at least one surfactant selected from the zwitterionic and/or amphoteric surfactants a total quantity of 0.01 to 5.0 wt. %.

3. The hair treatment agent according to claim 1, wherein the cationic amino silicone is selected from the group consisting of Silicone Quaternium-1, Silicone Quaternium-2, Silicone Quaternium-3, Silicone Quaternium-4, Silicone Quaternium-5, Silicone Quaternium-6, Silicone Quaternium-7, Silicone Quaternium-8, Silicone Quaternium-9, Silicone Quaternium-10, Silicone Quaternium-11, Silicone Quaternium-12, Silicone Quaternium-15, Silicone Quaternium-16, Silicone Quaternium-17, Silicone Quaternium-18, Silicone Quaternium-20, Silicone Quaternium-21 and Silicone Quaternium-22.

4. The hair treatment agent according to claim 1, wherein the cationic amino silicone is Silicone Quaternium-22.

5. The hair treatment agent according to claim 1, further comprising at least one cationic surfactant in a total quantity of 0.1 to 10.0 wt. %, based on the weight of the overall composition, the at least one cationic surfactant being selected from the group consisting of
i) ester quats,
ii) quaternary imidazolines of the formula (Tkat2) [chemical structure]
in which the residues R independently of one another each denote a saturated or unsaturated, linear or branched hydrocarbon residue with a chain length of 8 to 30 carbon atoms and A denotes a physiologically acceptable anion,
iii) amines and/or cationized amines,
and mixtures thereof.

6. The hair treatment agent according to claim 2, wherein the zwitterionic and/or amphoteric surfactant is cocamidopropyl betaine and/or coco-betaine.

7. The hair treatment agent according to claim 1, further comprising at least one active substance selected from the group consisting of carnitine, taurine, coenzyme Q-10, ectoine, a purine and derivatives and physiologically acceptable salts thereof, and a vitamin from the B series.

8. The hair treatment agent according to claim 5, wherein the cationic surfactant is selected from the group consisting of Stearamidopropyl Dimethylamine, Distearoylethyl Hydroxyethylmonium Methosulfate, Dicocoyl Hydroxyethylmonium Methosulfate, Dipalmitoylethyl Dimonium Chloride, Quaternium-27 Quaternium-91, and Behenoyl PG-Trimonium Chloride.

9. A method for treating keratinic fibers, comprising:
applying a cosmetic composition according to claim 1 onto the keratinic fibers, and rinsing the cosmetic composition out after an exposure time of a few seconds to 45 minutes.
